SEQUENCE LISTING (14 sequences)

SEQ ID NO: 1 - DNA, 274 bp, Homo sapiens
  misc_feature (7)..(7): n = a, c, g, or t
cccgggntatgagcaccaatacaatggtgctgacatccatcaaaggcacaccactctatatgtctccagagctggtggaggagcgaccatacgaccacacagcggacctctggtctgttggctgcatactatatgaactggcagtaggcaaccctcccttctatgctncaagcatctttnaggtggtcagcctcattctcaaggaccctgtgcgctgggcctcaaccatcagtccctgctttaagnacttcctgcagggantgtcaccaaagac

SEQ ID NO: 2 - DNA, 251 bp, Homo sapiens
  misc_feature (167)..(167): n = a, c, g, or t
tgaggagcttggagctcctaagctgggggcgtcccagatgttgtggaactgtccctggatctatagctcttcaccgtctctactttcttccttctaagagatcctgaaacctctgtcatggaaaagtaccacgtgttggagatgattggagaaggctcttttggganggtgtacaagggtcgaagaaaatacagtgctcaggtcgtggccctgaagttcatcccaaaattngggcgctcagagaaggagct

SEQ ID NO: 3 - DNA, 4845 bp, Homo sapiens
ggctcgagtgaggagcttggagctcctaggctgggggcgtcccaaatgttgtggaactgtccctggatctatagctcttcaccgtctctactttcttccttctaagagatcctgaaacctctgtcatggaaaagtaccacgtgttggagatgattggagaaggctcttttgggagggtgtacaagggtcgaagaaaatacagtgctcaggtcgtggccctgaagttcatcccaaaattggggcgctcagagaaggagctgaggaatttgcaacgagagattgaaataatgcggggtctgcggcatcccaacattgtgcatatgcttgacagctttgaaactgataaagaggtggtggtggtgacagactatgctgagggagagctctttcagatcctagaagatgacggaaaacttcctgaagaccaggttcaggccattgctgcccagttggtgtcagccctgtactatctgcattcccaccgcatcctacaccgagatatgaagcctcagaacatcctcctcgccaagggtggtggcatcaagctctgtgactttggatttgcccgggctatgagcaccaatacaatggtgctgacatccatcaaaggcacaccactctatatgtctccagagctggtggaggagcgaccatacgaccacacagcggacctctggtctgttggctgcatactatatgaactggcagtaggcacccctcccttctatgctacaagcatctttcagctggtcagcctcattctcaaggaccctgtgcgctggccctcaaccatcagtccctgctttaagtaataactgagccagcaggcccagatttggggaccccattcaccagccgcctacccccagaacttcaggtcctaaaggacgaacaggcccatcggttggcccccaagggtaatcagtctcgcatcttgactcaggcctataaacgcatggctgaggaggccatgcagaagaaacatcagaacacaggacctgcccttgagcaagaggacaagaccagcaaggtggctcctggcacagcccctctgcccagactcggggccactcctcaggaatcaagcctcctggccgggatcttagcctcagaattgaagagcagctgggctaaatcagggactggagaggtgccctctgcacctcgggaaaaccggaccaccccagattgtgaacgagcattcccagaggagaggccagaggtgctgggccagcggagcactgatgtagtggacctggaaaatgaggagccagacagtgacaatgagtggcagcacctgctagagaccactgagcctgtgcctattcaactgaaggctcctctcaccttgctgtgtaatcctgacttctgccagcgcatccagagtcagctgcatgaagctggagggcaggtgaagggctggagaagggagggagcagatgggaatggcacttagtctgacagagatcctgaggagaggcccctatccgttggtcactggtgttctgtgttagatcctgaaaggcatcttggagggtgcttcccacatcctgcctgcattccgggtcctgagcagtcttctctccagctgcagtgattctgttgccttgtattccttctgccgggaggcagggcttcctgggctgctgctgagtctactcaggcacagtcaggagagcaacagcctccagcagcaatcttggtatgggaccttcttacaggacctgatggctgtgattcaggcctactttgcctgtaccttcaatctggagaggagccagacaagtgacagcctgcaggtgtttcaggaggctgccaacctttttctggacctgttggggaaactgctggcccaaccagatgactctgagcagactttgcggagggacagccttatgtgctttactgtcctgtgcgaagccatggatgggaacagccgggccatctccaaagccttttactccagcttgctgacgacacagcaggttgtcttggatgggctccttcatggcttgacagttccacagctccctgtccacactccccaaggagccccgcaagtgagccagccactgcgagagcagagtgaggatatacctggagccatttcctctgccctggcagccatatgcactgctcctgtgggactgcccgactgctgggatgccaaggagcaggtctgttggcatttggcaaatcagctaactgaagacagcagccagctcaggccatccctcatctctggcctgcagcatcccatcctgtgcctgcaccttctcaaggttctatactcctgctgccttgtcagtgagggcctgtgccgtcttctggggcaggagcccctggccttggaatccctgtttatgttgattcagggcaaggtaaaagtagtagattgggaagagtctactgaagtgacactctacttcctctcccttcttgtctttcggcttcaaaacctgccttgtggaatggagaagctaggcagtgacgttgctactctctttacccattcgcatgtcgtctctcttgtgagtgcagcagcctgtctattgggacagcttggtcagcaaggggtgacctttgacctccagcccatggaatggatggctgcagccacacatgccttgtctgcccctgcagaggttcggttgactccaccaggtagttgtggattctatgatggcctccttatccttctgttgcagctcctcactgagcaggggaaggctagcctaatcagggatatgtccagttcagaaatgtggaccgttttgtggcaccgcttctccatggtcctgaggctccccgaggaggcatctgcacaggaaggggagctttcgctatccagtccaccaagccctgagccagactggacactgatttctccccagggcatggcagccctgctgagcctggccatggccacctttacccaggagccccagttatgcctgagctgcctgtcccagcatggaagtatcctcatgtccatcctgaagcatctgctttgccccagcttcctgaatcaactgcgccaggcgcctcatgggtctgagtttctccctgtcgtggtgctctctgtctgccagctcctttgcttcccctttgcgctggacatggatgctgacctccttataggtgtcttggccgacctcagggactcagaagttgcagcccatctgctgcaggtctgctgctaccatcttccgttgatgcaagtggagctgcccatcagccttctcacacgcctggccctcatggatcccacctctctcaaccagtttgtgaacacagtgtctgcctcccctagaaccatcgtctcgtttctctcagttgccctcctgagtgaccagccactgttgacctccgaccttctctctctgctggcccatactgccagggtcctgtctcccagccacttgtcctttatccaagagcttctggctggctctgatgaatcctatcggcccctgcgcagcctcctgggccacccagagaattctgtgcgggcacacacttataggctcctgggacacttgctccaacacagcatggccctgcgtggggcactgcagagccagtctggactgctcagccttctgctgcttgggcttggagacaaggatcctgttgtgcggtgcagtgccagctttgctgtgggcaatgcagcctaccaggctggtcctctgggacctgccctggcagctgcagtgcccagtatgacccagctgcttggagatcctcaggctggtatccggcgcaatgttgcatcagctctgggcaacttgggacctgaaggtttgggagaggagctgttacagtgcgaagtaccccagcggctcctagaaatggcatgtggagacccccagccaaatgtgaaggaggctgccctcattgccctccggagcctgcaacaggagcctggcatccatcaggtactggtgtccctgggtgccagtgagaaactatccttgctctctctggggaatcagtcactgccacacagcagtcctaggcctgcctctgccaaacactgcaggaaactcattcacctcctgaggccagcccatagcatgtgattccagattcctgcggtccagcctccaactttggttgccagctctttcttattctactacacaagccgccaactcaactgagagctaaagagactagaaaagagataagctgccaactcaactgagaacaagaaactagaagagatttatatataaagcttcttccttctcccagatgcaggatgttttcaaccagtaaattttattgctgttggtgccagagaagagtcctttcttctctacatccaggggccttttctccaataatgtgcctttaactctagggacctgcctcacggaccttagggaaaaacctcaacctgaaagatctcttcctttctggagctcctttaatcttcccagcaggtttttgccttagacgtgctggccccaggacagtgatgaagacagagcctgtctcagctctaggctgtggggatcaatgccatcagtccctgttattgagggattatcccttagccaacattcctatctgtgggtgggcgtggagagtgtatctttttttggggtgtgtgtgtatatgtgtgtgtgtatgtgtgtgtgtgtttaatagttctgtttgtaaactcttttaataaaagttgtgcctcaccatacttgaagctcccaggacaagggttgagaggctcaacccctctttcagcttctatgtggtgttggaggtgctggtatcgtgttcacacaaaaaaaaaaaaaaaaaaaaaaaaaaaaaaaaaaaaaaaaaaaaaaaaaaaaaaaaaaa

SEQ ID NO: 4 - DNA, 4237 bp, Homo sapiens
gcccgggctatgagcaccaatacaatggtgctgacatccatcaaaggcacaccactctatatgtctccagagctggtggaggagcgaccatacgaccacacagcggacctctggtctgttggctgcatactatatgaactggcagtaggcacccctcccttctatgctacaagcatctttcagctggtcagcctcattctcaaggaccctgtgcgctggccctcaaccatcagtccctgctttaagaacttcctgcagggactgctcaccaaagacccacggcagcgactgtcctggccagacctcttatatcacccctttattgctggtcatgtcaccataataactgagccagcaggcccagatttggggaccccattcaccagccgcctacccccagaacttcaggtcctaaaggacgaacaggcccatcggttggcccccaagggtaatcagtctcgcatcttgactcaggcctataaacgcatggctgaggaggccatgcagaagaaacatcagaacacaggacctgcccttgagcaagaggacaagaccagcaaggtggctcctggcacagcccctctgcccagactcggggccactcctcaggaatcaagcctcctggccgggatcttagcctcagaattgaagagcagctgggctaaatcagggactggagaggtgccctctgcacctcgggaaaaccggaccaccccagattgtgaacgagcattcccagaggagaggccagaggtgctgggccagcggagcactgatgtagtggacctggaaaatgaggagccagacagtgacaatgagtggcagcacctgctagagaccactgagcctgtgcctattcaactgaaggctcctctcaccttgctgtgtaatcctgacttctgccagcgcatccagagtcagctgcatgaagctggagggcagatcctgaaaggcatcttggagggtgcttcccacatcctgcctgcattccgggtcctgagcagtcttctctccagctgcagtgattctgttgccttgtattccttctgccgggaggcagggcttcctgggctgctgctgagtctactcaggcacagtcaggagagcaacagcctccagcagcaatcttggtatgggaccttcttacaggacctgatggctgtgattcaggcctactttgcctgtaccttcaatctggagaggagccagacaagtgacagcctgcaggtgtttcaggaggctgccaacctttttctggacctgttggggaaactgctggcccaaccagatgactctgagcagactttgcggagggacagccttatgtgctttactgtcctgtgcgaagccatggatgggaacagccgggccatctccaaagccttttactccagcttgctgacgacacagcaggttgtcttggatgggctccttcatggcttgacagttccacagctccctgtccacactccccaaggagccccgcaagtgagccagccactgcgagagcagagtgaggatatacctggagccatttcctctgccctggcagccatatgcactgctcctgtgggactgcccgactgctgggatgccaaggagcaggtctgttggcatttggcaaatcagctaactgaagacagcagccagctcaggccatccctcatctctggcctgcagcatcccatcctgtgcctgcaccttctcaaggttctatactcctgctgccttgtcagtgagggcctgtgccgtcttctggggcaggagcccctggccttggaatccctgtttatgttgattcagggcaaggtaaaagtagtagattgggaagagtctactgaagtgacactctacttcctctcccttcttgtctttcggcttcaaaacctgccttgtggaatggagaagctaggcagtgacgttgctactctctttacccattcgcatgtcgtctctcttgtgagtgcagcagcctgtctattgggacagcttggtcagcaaggggtgacctttgacctccagcccatggaatggatggctgcagccacacatgccttgtctgcccctgcagaggttcggttgactccaccaggtagttgtggattctatgatggcctccttatccttctgttgcagctcctcactgagcaggggaaggctagcctaatcagggatatgtccagttcagaaatgtggaccgttttgtggcaccgcttctccatggtcctgaggctccccgaggaggcatctgcacaggaaggggagctttcgctatccagtccaccaagccctgagccagactggacactgatttctccccagggcatggcagccctgctgagcctggccatggccacctttacccaggagccccagttatgcctgagctgcctgtcccagcatggaagtatcctcatgtccatcctgaagcatctgctttgccccagcttcctgaatcaactgcgccaggcgcctcatgggtctgagtttctccctgtcgtggtgctctctgtctgccagctcctttgcttcccctttgcgctggacatggatgctgacctccttataggtgtcttggccgacctcagggactcagaagttgcagcccatctgctgcaggtctgctgctaccatcttccgttgatgcaagtggagctgcccatcagccttctcacacgcctggccctcatggatcccacctctctcaaccagtttgtgaacacagtgtctgcctcccctagaaccatcgtctcgtttctctcagttgccctcctgagtgaccagccactgttgacctccgaccttctctctctgctggcccatactgccagggtcctgtctcccagccacttgtcctttatccaagagcttctggctggctctgatgaatcctatcggcccctgcgcagcctcctgggccacccagagaattctgtgcgggcacacacttataggctcctgggacacttgctccaacacagcatggccctgcgtggggcactgcagagccagtctggactgctcagccttctgctgcttgggcttggagacaaggatcctgttgtgcggtgcagtgccagctttgctgtgggcaatgcagcctaccaggctggtcctctgggacctgccctggcagctgcagtgcccagtatgacccagctgcttggagatcctcaggctggtatccggcgcaatgttgcatcagctctgggcaacttgggacctgaaggtttgggagaggagctgttacagtgcgaagtaccccagcggctcctagaaatggcatgtggagacccccagccaaatgtgaaggaggctgccctcattgccctccggagcctgcaacaggagcctggcatccatcaggtactggtgtccctgggtgccagtgagaaactatccttgctctctctggggaatcagtcactgccacacagcagtcctaggcctgcctctgccaaacactgcaggaaactcattcacctcctgaggccagcccatagcatgtgattccagattcctgcggtccagcctccaactttggttgccagctctttcttattctactacacaagccgccaactcaactgagagctaaagagactagaaaagagataagctgccaactcaactgagaacaagaaactagaagagatttatatataaagcttcttccttctcccagatgcaggatgttttcaaccagtaaattttattgctgttggtgccagagaagagtcctttcttctctacatccaggggccttttctccaataatgtgcctttaactctagggacctgcctcacggaccttagggaaaaacctcaacctgaaagatctcttcctttctggagctcctttaatcttcccagcaggtttttgccttagacgtgctggccccaggacagtgatgaagacagagcctgtctcagctctaggctgtggggatcaatgccatcagtccctgttattgagggattatcccttagccaacattcctatctgtgggtgggcgtggagagtgtatctttttttggggtgtgtgtgtatatgtgtgtgtgtatgtgtgtgtgtgtttaatagttctgtttgtaaactcttttaataaaagttgtgcctcaccatacttgaagctcccaggacaagggttgagaggctcaacccctctttcagcttctatgtggtgttggaggtgctggtatcgtgttcacacaaaaaaaaaaaaaaaaaaaaaaa

SEQ ID NO: 5 - DNA, 1425 bp, Homo sapiens
  CDS (1)..(1425)
atggaaaagtaccacgtgttggagatgattggagaaggctcttttgggagggtgtacaagggtcgaagaaaatacagtgctcaggtcgtggccctgaagttcatcccaaaattggggcgctcagagaaggagctgaggaatttgcaacgagagattgaaataatgcggggtctgcggcatcccaacattgtgcatatgcttgacagctttgaaactgataaagaggtggtggtggtgacagactatgctgagggagagctctttcagatcctagaagatgacggaaaacttcctgaagaccaggttcaggccattgctgcccagttggtgtcagccctgtactatctgcattcccaccgcatcctacaccgagatatgaagcctcagaacatcctcctcgccaagggtggtggcatcaagctctgtgactttggatttgcccgggctatgagcaccaatacaatggtgctgacatccatcaaaggcacaccactctatatgtctccagagctggtggaggagcgaccatacgaccacacagcggacctctggtctgttggctgcatactatatgaactggcagtaggcacccctcccttctatgctacaagcatctttcagctggtcagcctcattctcaaggaccctgtgcgctggccctcaaccatcagtccctgctttaagaacttcctgcagggactgctcaccaaagacccacggcagcgactgtcctggccagacctcttatatcacccctttattgctggtcatgtcaccataataactgagccagcaggcccagatttggggaccccattcaccagccgcctacccccagaacttcaggtcctaaaggacgaacaggcccatcggttggcccccaagggtaatcagtctcgcatcttgactcaggcctataaacgcatggctgaggaggccatgcagaagaaacatcagaacacaggacctgcccttgagcaagaggacaagaccagcaaggtggctcctggcacagcccctctgcccagactcggggccactcctcaggaatcaagcctcctggccgggatcttagcctcagaattgaagagcagctgggctaaatcagggactggagaggtgccctctgcacctcgggaaaaccggaccaccccagattgtgaacgagcattcccagaggagaggccagaggtgctgggccagcggagcactgatgtagtggacctggaaaatgaggagccagacagtgacaatgagtggcagcacctgctagagaccactgagcctgtgcctattcaactgaaggctcctctcaccttgctgtgtaatcctgacttctgccagcgcatccagagtcagctgcatgaagctggagggcaggtgaagggctggagaagggagggagcagatgggaatggcacttag

SEQ ID NO: 6 - protein, 474 aa, Homo sapiens [sequence omitted]

SEQ ID NO: 7 - DNA, 3948 bp, Homo sapiens
  CDS (1)..(3948)
atggaaaagtaccacgtgttggagatgattggagaaggctcttttgggagggtgtacaagggtcgaagaaaatacagtgctcaggtcgtggccctgaagttcatcccaaaattggggcgctcagagaaggagctgaggaatttgcaacgagagattgaaataatgcggggtctgcggcatcccaacattgtgcatatgcttgacagctttgaaactgataaagaggtggtggtggtgacagactatgctgagggagagctctttcagatcctagaagatgacggaaaacttcctgaagaccaggttcaggccattgctgcccagttggtgtcagccctgtactatctgcattcccaccgcatcctacaccgagatatgaagcctcagaacatcctcctcgccaagggtggtggcatcaagctctgtgactttggatttgcccgggctatgagcaccaatacaatggtgctgacatccatcaaaggcacaccactctatatgtctccagagctggtggaggagcgaccatacgaccacacagcggacctctggtctgttggctgcatactatatgaactggcagtaggcacccctcccttctatgctacaagcatctttcagctggtcagcctcattctcaaggaccctgtgcgctggccctcaaccatcagtccctgctttaagaacttcctgcagggactgctcaccaaagacccacggcagcgactgtcctggccagacctcttatatcacccctttattgctggtcatgtcaccataataactgagccagcaggcccagatttggggaccccattcaccagccgcctacccccagaacttcaggtcctaaaggacgaacaggcccatcggttggcccccaagggtaatcagtctcgcatcttgactcaggcctataaacgcatggctgaggaggccatgcagaagaaacatcagaacacaggacctgcccttgagcaagaggacaagaccagcaaggtggctcctggcacagcccctctgcccagactcggggccactcctcaggaatcaagcctcctggccgggatcttagcctcagaattgaagagcagctgggctaaatcagggactggagaggtgccctctgcacctcgggaaaaccggaccaccccagattgtgaacgagcattcccagaggagaggccagaggtgctgggccagcggagcactgatgtagtggacctggaaaatgaggagccagacagtgacaatgagtggcagcacctgctagagaccactgagcctgtgcctattcaactgaaggctcctctcaccttgctgtgtaatcctgacttctgccagcgcatccagagtcagctgcatgaagctggagggcagatcctgaaaggcatcttggagggtgcttcccacatcctgcctgcattccgggtcctgagcagtcttctctccagctgcagtgattctgttgccttgtattccttctgccgggaggcagggcttcctgggctgctgctgagtctactcaggcacagtcaggagagcaacagcctccagcagcaatcttggtatgggaccttcttacaggacctgatggctgtgattcaggcctactttgcctgtaccttcaatctggagaggagccagacaagtgacagcctgcaggtgtttcaggaggctgccaacctttttctggacctgttggggaaactgctggcccaaccagatgactctgagcagactttgcggagggacagccttatgtgctttactgtcctgtgcgaagccatggatgggaacagccgggccatctccaaagccttttactccagcttgctgacgacacagcaggttgtcttggatgggctccttcatggcttgacagttccacagctccctgtccacactccccaaggagccccgcaagtgagccagccactgcgagagcagagtgaggatatacctggagccatttcctctgccctggcagccatatgcactgctcctgtgggactgcccgactgctgggatgccaaggagcaggtctgttggcatttggcaaatcagctaactgaagacagcagccagctcaggccatccctcatctctggcctgcagcatcccatcctgtgcctgcaccttctcaaggttctatactcctgctgccttgtcagtgagggcctgtgccgtcttctggggcaggagcccctggccttggaatccctgtttatgttgattcagggcaaggtaaaagtagtagattgggaagagtctactgaagtgacactctacttcctctcccttcttgtctttcggcttcaaaacctgccttgtggaatggagaagctaggcagtgacgttgctactctctttacccattcgcatgtcgtctctcttgtgagtgcagcagcctgtctattgggacagcttggtcagcaaggggtgacctttgacctccagcccatggaatggatggctgcagccacacatgccttgtctgcccctgcagaggttcggttgactccaccaggtagttgtggattctatgatggcctccttatccttctgttgcagctcctcactgagcaggggaaggctagcctaatcagggatatgtccagttcagaaatgtggaccgttttgtggcaccgcttctccatggtcctgaggctccccgaggaggcatctgcacaggaaggggagctttcgctatccagtccaccaagccctgagccagactggacactgatttctccccagggcatggcagccctgctgagcctggccatggccacctttacccaggagccccagttatgcctgagctgcctgtcccagcatggaagtatcctcatgtccatcctgaagcatctgctttgccccagcttcctgaatcaactgcgccaggcgcctcatgggtctgagtttctccctgtcgtggtgctctctgtctgccagctcctttgcttcccctttgcgctggacatggatgctgacctccttataggtgtcttggccgacctcagggactcagaagttgcagcccatctgctgcaggtctgctgctaccatcttccgttgatgcaagtggagctgcccatcagccttctcacacgcctggccctcatggatcccacctctctcaaccagtttgtgaacacagtgtctgcctcccctagaaccatcgtctcgtttctctcagttgccctcctgagtgaccagccactgttgacctccgaccttctctctctgctggcccatactgccagggtcctgtctcccagccacttgtcctttatccaagagcttctggctggctctgatgaatcctatcggcccctgcgcagcctcctgggccacccagagaattctgtgcgggcacacacttataggctcctgggacacttgctccaacacagcatggccctgcgtggggcactgcagagccagtctggactgctcagccttctgctgcttgggcttggagacaaggatcctgttgtgcggtgcagtgccagctttgctgtgggcaatgcagcctaccaggctggtcctctgggacctgccctggcagctgcagtgcccagtatgacccagctgcttggagatcctcaggctggtatccggcgcaatgttgcatcagctctgggcaacttgggacctgaaggtttgggagaggagctgttacagtgcgaagtaccccagcggctcctagaaatggcatgtggagacccccagccaaatgtgaaggaggctgccctcattgccctccggagcctgcaacaggagcctggcatccatcaggtactggtgtccctgggtgccagtgagaaactatccttgctctctctggggaatcagtcactgccacacagcagtcctaggcctgcctctgccaaacactgcaggaaactcattcacctcctgaggccagcccatagcatgtga

SEQ ID NO: 8 - protein, 1315 aa, Homo sapiens
MEKYHVLEMIGEGSFGRVYKGRRKYSAQVVALKFIPKLGRSEKELRNLQREIEIMRGLRHPNIVHMLDSFETDKEVVVVTDYAEGELFQILEDDGKLPEDQVQAIAAQLVSALYYLHSHRILHRDMKPQNILLAKGGGIKLCDFGFARAMSTNTMVLTSIKGTPLYMSPELVEERPYDHTADLWSVGCILYELAVGTPPFYATSIFQLVSLILKDPVRWPSTISPCFKNFLQGLLTKDPRQRLSWPDLLYHPFIAGHVTIITEPAGPDLGTPFTSRLPPELQVLKDEQAHRLAPKGNQSRILTQAYKRMAEEAMQKKHQNTGPALEQEDKTSKVAPGTAPLPRLGATPQESSLLAGILASELKSSWAKSGTGEVPSAPRENRTTPDCERAFPEERPEVLGQRSTDVVDLENEEPDSDNEWQHLLETTEPVPIQLKAPLTLLCNPDFCQRIQSQLHEAGGQILKGILEGASHILPAFRVLSSLLSSCSDSVALYSFCREAGLPGLLLSLLRHSQESNSLQQQSWYGTFLQDLMAVIQAYFACTFNLERSQTSDSLQVFQEAANLFLDLLGKLLAQPDDSEQTLRRDSLMCFTVLCEAMDGNSRAISKAFYSSLLTTQQVVLDGLLHGLTVPQLPVHTPQGAPQVSQPLREQSEDIPGAISSALAAICTAPVGLPDCWDAKEQVCWHLANQLTEDSSQLRPSLISGLQHPILCLHLLKVLYSCCLVSEGLCRLLGQEPLALESLFMLIQGKVKVVDWEESTEVTLYFLSLLVFRLQNLPCGMEKLGSDVATLFTHSHVVSLVSAAACLLGQLGQQGVTFDLQPMEWMAAATHALSAPAEVRLTPPGSCGFYDGLLILLLQLLTEQGKASLIRDMSSSEMWTVLWHRFSMVLRLPEEASAQEGELSLSSPPSPEPDWTLISPQGMAALLSLAMATFTQEPQLCLSCLSQHGSILMSILKHLLCPSFLNQLRQAPHGSEFLPVVVLSVCQLLCFPFALDMDADLLIGVLADLRDSEVAAHLLQVCCYHLPLMQVELPISLLTRLALMDPTSLNQFVNTVSASPRTIVSFLSVALLSDQPLLTSDLLSLLAHTARVLSPSHLSFIQELLAGSDESYRPLRSLLGHPENSVRAHTYRLLGHLLQHSMALRGALQSQSGLLSLLLLGLGDKDPVVRCSASFAVGNAAYQAGPLGPALAAAVPSMTQLLGDPQAGIRRNVASALGNLGPEGLGEELLQCEVPQRLLEMACGDPQPNVKEAALIALRSLQQEPGIHQVLVSLGASEKLSLLSLGNQSLPHSSPRPASAKHCRKLIHLLRPAHSM

SEQ ID NO: 9 - DNA, 20 bp, Homo sapiens
tcaggcctataaacgcatgg

SEQ ID NO: 10 - DNA, 19 bp, Homo sapiens
cacctgcaggctgtcactt

SEQ ID NO: 11 - DNA, 20 bp, Homo sapiens
gtggaggagcgaccatacga

SEQ ID NO: 12 - DNA, 23 bp, Homo sapiens
cctttaggtcctgaagttctggg

SEQ ID NO: 13 - DNA, 19 bp, Homo sapiens
cagcgcatccagagtcagc

SEQ ID NO: 14 - DNA, 20 bp, Homo sapiens
ccggcagaaggaatacaagg

(19) United States
(12) Patent Application Publication
(10) Pub. No.: US 2006/0084600 A1
(43) Pub. Date: Apr. 20, 2006

(54) Human homologues of fused gene

(75) Inventors: Monica Mosca, Milan (IT); Antonella Isacchi, Milan (IT)

Correspondence Address: AKIN, GUMP, STRAUSS, HAUER & FELD, 1111 LOUISIANA STREET, 44TH FLOOR, HOUSTON, TX, 77002, US

(73) Assignee: HSB Solomon Associates, LLC, Dallas, TX (US)

(21) Appl. No.: 11/086,960

(22) Filed: Mar. 21, 2005

Related U.S. Application Data
(63) Continuation of application No. 09/958,946, filed on Oct. 16, 2001, now abandoned.

(30) Foreign Application Priority Data
Apr. 16, 1999 (GB) 9908798.3

(51) Int. Cl. A61K 38/17; C07K 14/705; C07H 21/04; C12P 21/04

(52) U.S. Cl. 514/12; 435/69.7; 435/320.1; 435/325; 530/350; 536/23.5; 435/456

(57) ABSTRACT

The invention relates to novel molecules, in particular nucleic acid sequences and the encoded polypeptides, which are the human homologues of the protein-serine/threonine kinase named Fused.

### FIELD OF THE INVENTION

This invention relates to novel molecules, in particular nucleic acid sequences and the encoded polypeptides, which are the human homologues of the protein-serine/threonine kinase named Fused. The molecules of the inventions are involved in the transduction of the signal in the Hedgehog-Patched (HH-PTCH) pathway.

### BACKGROUND OF THE INVENTION

The Hedgehog-Patched (HH-PTCH) signalling pathway is functionally homologue to the Drosophila melanogaster Hedgehog-Patched (HH-PTC) pathway, which plays a critical role in determining cell fate, pattering and differentiation during embryonic development.

The evolution introduced a diversification of the numbers of genes involved in the Hedgehog-Patched signalling and in vertebrates three major families of hedgehog genes, which regulate the development, have been identified: Indian hedgehog (Ihh), Desert hedgehog (Dhh) and Sonic hedgehog (Shh).

The Sonic hedgehog (Shh) pathway co-ordinates anterior/posterior pattering of the limb during vertebrate embriogenesis and its gene expression is involved in the control of the somite pattering, progenitors of the sclerotome and dermamyotome.

Moreover Sonic hedgehog expression directs the development of neurons in the ventral neural tube while in the central nervous system it induces the differentiation of the midbrain dopaminergic (DA) neurons.

Indian hedgehog (Ihh) is expressed in the developing cartilage elements and it indirectly maintains prehypertrophic chondrocyte in proliferative state; Desert hedgehog (Dhh) is expressed in the Sertoli cell precursors and interestingly it remains expressed in the testis even during adult life.

The critical role of the HH-PTCH pathway in tissue differentiation is confirmed by the finding that mutations of the patched (ptc) gene disrupt the polarity of the segments in the Drosophila embryo while patched deficient mice (ptch) die before birth due to multiple development defects.

The Patched (PTC) protein is a transmembrane receptor, which acts as a constitutive repressor of the transmembrane protein Smoothened (SMO).

Upon binding of its ligand Hedgehog (HH) the PTC, protein undergoes a conformational change thus releasing its negative regulation on SMO, allowing it to transmit a signal to the nucleus, where it activates specific transcriptional factors. It can be derived that PTC acts like a constitutive repressor and mutations of the ptc gene result in the abrogation of a negative feedback loop on the transcription of the HH regulated genes such as ptc itself.

As a matter of fact, in Drosophila ptc mutant cells, the HH-regulated genes are overexpressed.

Actually the Patched pathway is largely unknown even if in Drosophila several proteins involved in the transduction of the intracellular signals had been identified such as the protein-serine/threonine kinase Fused (FU) (GenBank Acc X80468), a protein with unknown biochemical function named Suppressor of Fused (SU(FU)), the distant relative of the kinesin motor proteins Costal2 (COS2), the zinc transcription factors Ci.

SU(FU) acts as a bridge between FU and Ci and the trimeric complex prevents the proteolytic cleavage of SU(FU) while, on the other hand, the activation of the serine/threonine kinase FU triggers the degradation of SU(FU) through phosphorylation of its PEST sequence (sequences enriched in proline (P), glutamic acid (E), serine (S) and threonine(T)).

The human homologue of PTC was isolated (PTCH1) (Johnson, R. L. et al. (1996) Science 272:1668-1671) and recently different splicing forms encoding the PTCH2 receptor had been identified. The two patched receptors (PTCH1. PTCH2) seem to have distinct functions since up-regulation Of ptch2 expression is unable to compensate for inactive PTCH1 protein. Moreover, in mouse embryo, the expression patterns of ptch1 and ptch2 do not fully overlap (Carpenter D. et al (1998) P.N.A.S. 95:13630-13634 and Zaphiropopulos P. et al (1999) Canc. Res. 59: 787-792).

While the role of the Ptch pathway in human adult tissues is still poorly understood, genetic studies have provided evidence that germline mutations and/or deletions of the ptch1 gene are involved in the Nevoid Basal Cell Carcinoma Syndrome (NBCCS) (Heidi Hahn et al. (1996) Cell 85:814-851).

NBCCS is an autosomal dominant disorder that predisposes to basal cell carcinomas (BCCs) of the skin and medulloblastomas. NBCCS patients share an increased risk for ovarian fibromas, meningiomas, fibrosarcomas, rhabdomyosarcomas and a range of developmental abnormalities primarily affecting the limbs and the axial skeleton.

Since the mutations of the ptch1 gene are straightforward related to the NBCCS disorder, characterized by a strong predisposition to cancer, this evidence points to the conclusion that the abrogation of the ptch1 negative feedback loop plays a fundamental role in tumour development. Indeed, in tumour cells of both familial and sporadic BOCs the genes controlled by Shh, such as ptch1 (Unden et al. Cancer Res (1997), 57:2336-2340) and gli1 (Dahmane et al Nature (1997), 369: 876-881), are transcriptionally derepressed.

Therefore, it is of utmost importance to investigate the function of the SHH-PTCH signalling pathway in normal adult tissues and in major forms of human cancer, besides BCCs.

To achieve this goal the human homologue of the genes involved in the transduction of the signals need to be isolated and sequenced.

Genetic analysis suggest the activation of the serine/threonine kinase Fused (FU) as the result of the HH activity. These results are in agreement with biochemical evidences which show that FU is phosphorylated during the signalling transduction events initiated by HH.

Since mutations of the segment polarity gene Fused (FU) are strikingly correlated to the alteration of the Drosophila embryo phenotype, then it is highly likely that Fused (hFU) plays a key function in the development of human tumours as well. Therefore the human homologue of fused (hFU) need to be isolated to gain information about the role of the hFU kinase in the regulation of the expression of the genes induced by Shh.

The present invention achieved this goal by providing the characterisation and the nucleic acid sequences encoding two novel human homologues of the Fused protein.

### SUMMARY OF THE INVENTION

In a first aspect, the present invention provides isolated polynucleotides comprising a sequence that encodes a human homologue of the Fused protein (hFU).

In another aspect, the polynucleotide of the present invention is contained in an expression vector.

The present invention still further provides for a host cell transformed with an expression vector of this invention.

In a still further aspect the invention provides isolated and purified recombinant hFU proteins which are coded for by the polynucleotides of the invention.

Furthermore, the invention provides nucleic acid probes whose sequence are derived from the polynucleotides of the invention; these probes can be used as research tools as well as in diagnostic methods, for example to detect and measure hFU biosynthesis in tissues and cells.

Accordingly, it is an object of the present invention to provide a diagnostic method for detecting the presence and the amount of hFU in tissues and cells.

The present invention also includes screening methods for identifying a compound capable of interacting with hFU. The screening method can be in any configuration well known to those skilled in the art.

It is a further object of the present invention to provide compounds identified with the said screening method and capable of modulating the biological activity of hFU.

It is still a further object of the invention to provide antibodies directed against epitopes present in hFU as well as cells producing the antibody.

In a further object the invention includes a recombinant process for the expression of hFU, which process comprises PCR amplification, cloning, expression and purification of the recombinant enzyme.

Definitions

As used herein the following terms have the meaning defined below.

The abbreviation for human fused in lower case (hfu) refers to a gene, cDNA, RNA or nucleic acid sequence while the upper case version (hFU) refers to a protein, polypeptide, peptide, oligopeptide, or amino acid sequence.

As used herein, the term “analogous” for nucleotide sequences refers to those nucleotide sequences that encode analogous polypeptides, analogous polypeptides being those which have only conservative differences and which retain the conventional characteristics and activities of the hFU homologues of the invention.

The term “stringent conditions” relates to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion (or other salts) at pH 7.0 to 8.3 and the temperature is at least about 30° C. for short probes (e.g. 10 to 50 nucleotides) and at least about 60° C. for longer probes. Stringent conditions may also be achieved with the addition of destabilizing agents, such as formamide.

“Homologous nucleotide sequences” are those sequences characterized by an homology, at the nucleotide level, greater than about 60%, preferably greater than about 70%, more preserably greater than about 80%, in at least one functional domain of the encoded polypeptide, with respect to the corresponding region of the sequences disclosed in the present invention. Homologous nucleotide sequences include those sequences coding for isoforms of the hFU proteins encoded by the DNA sequences set forth below. Such isoforms can be expressed in different tissues of the same organism as a result, e.g., of alternative splicing or because coded for by other genes. Homologous nucleotide sequences are also those sequences coding for a hFU protein of any species of origin. Preferably the nucleotide sequences code for enzymes of mammalian origin.

An “oligonucleotide” is a stretch of nucleotide residues which has a sufficient number of bases to be used in a polymerase chain reaction (PCR). This short sequence is based on (or designed from) a genomic or cDNA sequence and is used to amplify, confirm, or reveal the presence of an identical, similar or complementary DNA or RNA in a particular cell or tissue. Oligonucleotides comprise portions of a DNA sequence having at least about 10 nucleotides and as many as about 50 nucleotides, preferably about 15 to 30 nucleotides. They are chemically synthesized and may be used as probes.

“Probes” are nucleic acid sequences of variable length, preferably between at least about 10 and as many as about 6,000 nucleotides, depending on use. They are used in the detection of identical, similar, or complementary nucleic acid sequences. Longer length probes are usually obtained from a natural or recombinant source, are highly specific and much slower to hybridize than oligomers. They may be single- or double-stranded and carefully designed to have specificity in PCR, hybridization membrane-based, or ELISA-like technologies.

Amino acid “insertions”, “substitutions” or “deletions” are changes to or within an amino acid sequence. The variation allowed in a particular amino acid sequence may be experimentally determined by producing the peptide synthetically or by systematically making insertions, deletions, or substitutions of nucleotides in the hFU sequence using recombinant DNA techniques.

In the present application we refer to the BLAST algorithm. The BLAST algorithm is suitable for determining sequence similarity and it is described in Altschul et al. (1990) J. Mol. Biol. 215: 403-410. Software for performing BLAST analyses is publicly available through the National Center for Biotechnology Information (httd://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extension for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The Blast algorithm parameters W, T and X determine the sensitivity and speed of the alignment. The Blast program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a fused gene or cDNA if the smallest sum probability in comparison of the test nucleic acid to a fused nucleic acid is less than about 1, preferably less than about 0.1, more preferably less than about 0.01, and most preferably less than about 0.001.

### DETAILED DESCRIPTION OF THE INVENTION

For the purposes of the present invention as disclosed and claimed herein, the following is to be considered.

The amino acid sequences are presented in the amino to carboxy direction, from left to right. The amino and carboxy groups are not presented in the sequence.

The nucleotide sequences are presented by single strand only, in the 5′ to 3′ direction, from left to right.

Nucleotides and amino acids are represented in the manner recommended by the IUPAC-IUB Biochemical Nomenclature Commission, or (for amino acids) by three letters code.

In one aspect, the present invention provides isolated and purified polynucleotides that encode two novel human homologues of the Fused proteins (hFU). A polynucleotide of the present invention is an isolated single or double stranded polynucleotide which comprises a sequence selected from the group consisting of:

(a) the sense sequence of SEQ ID NO. 5 and the sense sequence of SEQ ID NO.7;

(b) sequences complementary to the sequences of (a);

(c) a sequence that encodes a polypeptide encoded by the sequences of (a);

(d) analogous sequences that hybridize under stringent conditions to the sequences of (a) and (b); and

(e) sequences that are homologous to the sequences of (a) and that encode polypeptides having hFU activity.

A preferred polynucleotide is a DNA molecule. In another embodiment, the polynucleotide is an RNA molecule.

The nucleotide sequence of SEQ ID No. 5 represents a cDNA sequence of the sense strand of the gene encoding one of the hFU homologues and is intended to represent both the sense strand (shown on top) and its complementary strand. The nucleotide sequence of SEQ ID NO. 7 represents a cDNA sequence of the sense strand of the gene encoding another of the hFU homologues and is intended to represent both the sense strand (shown on top) and its complementary strand.

The present invention also contemplates analogous DNA sequences which hybridize under stringent conditions to the DNA sequences set forth above. The present invention also contemplates naturally occurring allelic variations and mutations of the DNA sequences set forth above so long as those variations and mutations code, on expression, for a human hFU homologue. Moreover, the invention contemplates homologous DNA sequences that, on expression, encode polypeptides having hFU activity (see definitions above).

As is well known in the art, because of the degeneracy of the genetic code, there are numerous other DNA and RNA molecules that can code for the same polypeptides as those encoded by the aforementioned hfu genes. The present invention, therefore, contemplates those other DNA and RNA molecules which, on expression, encode the polypeptides of SEQ ID NO. 6 and SEQ ID NO.8.

Having identified the amino acid residue sequences encoded by a hfu gene, and with knowledge of all triplet codons for each particular amino acid residue, it is possible to describe all such encoding RNA and DNA sequences. DNA and RNA molecules other than those specifically disclosed herein characterized simply by a change in a codon for a particular amino acid, are within the scope of this invention.

A table of amino acids and their representative abbreviations, symbols and codons is set forth below in the following Table.

| Amino acid | Abbrev. | Symbol | Codon(s) |
|---|---|---|---|
| Alanine | Ala | A | GCA GCC GCG GCU |
| Cysteine | Cys | C | UGA UGU |
| Aspartic acid | Asp | D | GAC GAU |
| Glutainic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

As is well known in the art, codons constitute triplet sequences of nucleotides in mRNA molecules and, as such, are characterized by the base uracil (U) in place of base thymidine (T) (which is present in DNA molecules).

A simple change in a codon for the same amino acid residue within a polynucleotide will not change the structure of the encoded polypeptide.

The hFU homologues encoded by SEQ ID NO.5 and SEQ ID NO. 7 constitute a preferred embodiment of the present invention However, it is to be understood that the present invention includes proteins homologous to, and having essentially the same biological properties as, the proteins coded for by the nucleotide sequences herein disclosed. This definition is intended to encompass isoforms and natural allelic variants of the two hFU homologues herein disclosed. These variant forms may result, e.g., from alternative splicing or differential expression in different tissue of the same source organism. The variant forms may be characterized by, e.g., amino acid insertion, deletion or substitution. In this connection, a variant form having an amino acid sequence which has at least approximately 80% sequence homology, preferably approximately 90% sequence homology, more preferably approximately 95% sequence homology and most preferably approximately 98% sequence homology to SEQ ID Nos.6 or 8; is contemplated as being included in the present invention.

With the knowledge of the sequence information disclosed in the present invention, the expert in the art can identify and obtain DNA sequences which encode the proteins of the invention from different sources (i.e. different tissues or different organisms) through a variety of means well known to him and disclosed by, for example, Maniatis et al., Molecular cloning: a laboratory manual, Second Edition, Cold Spring Harbor Press, Cold Spring Harbor, N.Y. (1989).

For example DNA which encodes the proteins of the invention may be obtained by screening of mRNA, cDNA or genomic DNA with oligonucleotide probes generated from the sequence information of the hfu genes provided herein. Probes may be labeled with a detectable group such as a fluorescent group, a radioactive atom or a chemiluminescent group in accordance with known procedures and used in conventional hybridization assays, as described by, for example, Maniatis et al. Molecular cloning: a laboratory manual, Second Edition, Cold Spring Harbor Press, Cold Spring. Harbor, N.Y. (1989) The probes find another useful application in diagnostic methods, for example to detect and measure hFU biosynthesis in tissues and cells.

The sequence of hfu genes may alternatively be recovered by use of the polymerase chain reaction (PCR) procedure, with the PCR oligonucleotide primers produced from the sequences provide herein. See U.S. Pat. No. 4,683,195 to Mullis et al. and U.S. Pat. No. 4,683,202 to Mullis. The PCR reaction provides a method for selectively increasing the concentration of a particular nucleic acid sequence even when that sequence has not been previously purified and is present only in a single copy in a particular sample. The method can be used to amplify either single- or double-stranded DNA. The essence of the method involves the use of two oligonucleotides probes to serve as primers for the template-dependent, polymerase mediated replication of a desired nucleic acid molecule.

A wide variety of alternative cloning and in vitro amplification methodologies are well known to those skilled in the art. Examples of these techniques are e.g. found in Berger and Kimmel, Guide to Molecular Cloning Techniques, Methods in Enzymology 152 Academic Press, Inc., San Diego, Calif. (Berger).

In order to replicate the DNA sequence encoding the hFU homologues, this must be cloned in an appropriate vector. A vector is a replicable DNA construct.

Vectors are used herein either to amplify DNA encoding the hFU homologues and/or to express DNA which encodes the proteins of the invention. An expression vector is a replicable DNA construct in which a DNA sequence encoding one of the proteins of the invention is operably linked to suitable control sequences capable of effecting the expression of one of the two hFU homologues in a suitable host. DNA regions are operably linked when they are functionally related to each other. For example: a promoter is operably linked to a coding sequence if it controls the transcription of the sequence. Amplification vectors do not require expression control domains. All that is needed is the ability to replicate in a host, usually conferred by an origin of replication, and a selection gene to facilitate recognition of transformants.

DNA sequences encoding the proteins of the invention may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulation are disclosed by Maniatis et al. Molecular cloning: a laboratory manual, Second Edition, Cold Spring Harbor Press, Cold Spring Harbor, N.Y. (1989) and are well known in the art.

Expression of the cloned sequence occurs when the expression vector is introduced into an appropriate host cell. If a prokaryotic expression vector is employed, then the appropriate host cell would be any prokaryotic cell capable of expressing the cloned sequences, for example E. coli. Similarly, if an eukaryotic expression vector is employed, then the appropriate host cell would be any eukaryotic cell capable of expressing the cloned sequence. A yeast host may be employed, for example S. cerevisiae. Alternatively, insect cells may be used, in which case a baculovirus vector system may be appropriate. Another alternative host is a mammalian cell line, for example COS-1 cells.

The need for control sequences into the expression vector will vary depending upon the host selected and the transformation method chosen. Generally, control sequences include a transcriptional promoter, an optional operator sequence to control transcription, a sequence encoding suitable mRNA ribosomal binding, and sequences which control the termination of transcription and translation. Vectors useful for practicing the present invention include plasmids, viruses (including phages), retroviruses, and integrable DNA fragments (i.e. fragments integrable into the host genome by homologous recombination). The vectors replicate and function independently of the host genome, or may, in some instances, integrate into the genome itself.

Expression vectors should contain a promoter which is recognized by the host organism. The promoter sequences of the present invention may be either prokaryotic, eukaryotic or viral. Example of suitable prokaryotic sequences include the P_{R} and P_{L} promoters of bacteriophage lambda (The bacteriophage Lambda, Hershey, A. D., Ed., Cold Spring Harbor Press, Cold Spring Harbor, N.Y. (1973); Lambda II, Hendrix, R. W., Ed., Cold Spring Harbor Press, Cold Spring Harbor, N.Y. (1980)); the trp, recA, heat shock, and lacZ promoters of E. coli and the SV40 early promoter (Benoist, C. et al. nature 290: 304-310 (1981)).

As far as the Shine Dalgarno sequence is concerned, preferred examples of suitable regulatory sequences are represented by the Shine-Dalgarno of the replicase gene of the phage MS-2 and of the gene cII of bacteriophage lambda. The Shine-Dalgarno sequence may be directly followed by the DNA encoding the hFU homologues and results in the expression of the mature proteins of the invention.

Alternatively, the DNA encoding the proteins of the invention may be preceded by a DNA sequence encoding a carrier peptide sequence. In this case, a fusion protein is produced in which the N-terminus of one of the hFU homologues is fused to a carrier peptide, which may help to increase the protein expression levels and intracellular stability, and provide simple means of purification. A preferred carrier peptide includes one or tore of the IgG binding domains of protein A which are easily purified to Homogeneity by affinity chromatography e.g. on IgG-coupled Sepharcse. Alternatively, many vectors have the advantage of carrying a stretch of histidine residues that can be expressed at the N-terminal or C-terminal end of the target protein. Thus the protein of interest can be recovered by metal chelation chromatography. A DNA sequence encoding a recognition site for a proteolytic enzyme such as enterokinase, factor X, procollagenase or thrombine may immediately precede the sequence encoding the hFU homologues to permit cleavage of the fusion proteins to obtain the mature proteins of the invention.

Moreover, a suitable expression vector includes an appropriate marker which allows the screening of the transformed host cells. The transformation of the selected host is carried out using any one of the various techniques well known to the expert in the art and described in Maniatis et al. Molecular cloning: a laboratory manual, Second Edition, Cold Spring Harbor Press, Cold Spring Harbor, N.Y. (1989).

One further embodiment of the invention is a prokaryotic host cell transformed with the said expression vector and able to produce, under appropriate culture conditions, the proteins of the invention.

Moreover, cultures of cells derived from multicellular organisms are a desiderable host for the synthesis of the hFU homologues. In principal, any eukaryotic cell culture is workable, whether from vertebrate or invertebrate culture, including insect and mammalian cells. Propagation of such cells in cell culture has become a routine procedure. See Tissue Culture, Academic Press, Kruse and Patterson, eds. (19731. Examples of useful host cell lines are HeLa cells, CHO and COS cell lines. The transcriptional and translational control sequences in expression vectors to be used in transforming vertebrate and invertebrate cells are often provided by viral sources, for example, commonly used promoters are derived front Adenovirus 2, polyoma and SV40. See, e.g. U.S. Pat. No. 4,599,308.

An origin of replication may be provided either by construction of the vector to include an exogenous origin or may be provided by the host cell chromosomal replication mechanism. If the vector is integrated into the host cell chromosome, the latter may be sufficient.

Rather than using vectors which contains viral origins of replication, one can transform mammalian cells by the method of cotransformation with a selectable marker and DNA encoding the hFU homologues. An example of a suitable marker is dihydrofolate reductase (DHFR) or thymidine kinase. See U.S. Pat. No. 4,399,216.

The hFU homologues according to the invention can be used as antigens for raising antibodies against the same Consequently, the invention also encompasses an antibody which specifically binds a protein according to the invention. Such antibodies are useful for immunoassays, e.g. for the isolation of peptides or polypeptides.

The antibodies according to the invention may be monoclonal or polyclonal and include individual, allelic, strain or species variants, or fragments thereof, both in their naturally occurring (full-length) forms and recombinant forms. Additionally, the antibodies are raised to the present proteins in either their native configuration or in non-native configurations. Anti-idiotypic antibodies can also be generated. Many methods of making antibodies are known to persons skilled in the art. For techniques for preparing monoclonal antibodies, see e.g. Stiites et al (eds.), Basic and Clinical Immunology (4^{th} ed), Lange Medical, Publications, Los Altos, Calif., and references cited therein. For techniques that involve selection of libraries of recombinant antibodies in phage or similar vectors, see e.g. Huse et al. (1989) Science 246:1275-1281.

The hFU homologues according to the invention may be used in pharmaceutical preparations. Accordingly, the invention relates to a hFU homologue or antibody according to the invention for use in a method of treatment of the human or animal body by therapy as well as to the use of said molecules in the manufacture of a medicament for use in the treatment of basal cell carcinomas. The invention also relates to a pharmacological preparation comprising a molecule according to the invention together with a pharmaceutically acceptable carrier. The molecule used in a method of treatment of the human or animal body may be any one of the above described hFU homologues or antibodies as well as any novel substance identified in a screening method using the same and described above.

In another aspect the invention features a method for screening for an agent that selectively inhibits the hFU kinase activity. The method involves assaying a potential agent for the ability to inhibit the hFU kinase activity but not inhibit an activity of another kinase.

The agent to be screened may be of extracellular, intracellular, biologic or chemical origin. The hFU homologue employed in such a test may either be free in solution, attached to a solid support, such as agarose or plastic beads, microtiter wells, nylon or citrocellulose, borne on a cell surface or located intracellularly. One may measure, for example, the formation of complexes between hFU and the agent being tested. Alternatively, one can examine the diminution in complex formation between hFU and its substrate caused by the agent being tested.

The invention also provides a method for screening for a compound which can affect hFU signal transduction comprising contacting such a compound with an hFU homologue of the invention and assaying for the presence of a complex between the compound and the hFU homologue. In such assays, the hFU homologue is typically labelled. After suitable incubation, free hFU homologue is separated from that present in bound form, and the amount of free or uncomplexed label is a measure of the ability of the particular compound to bind to hFU.

The compounds identified through the screening method hereby described are within the scope of the present invention. Such compounds are preferably low molecular weight molecules of peptidic or non-peptidic nature. Thanks to their small size they are suitably used for medicinal purposes.

Another technique for drug screening provides high throughput screening for compounds having suitable binding affinity to the hFU homologue. Briefly stated, large numbers of different small peptide test compounds are synthesized on a solid substrate. The peptide test compounds are reacted with the hFU homologue and washed. Bound hFU homologue is then detected by methods well known in the art.

The hFU homologues of the invention may also be used to assay the ability of a test compound to inhibit the formation of the complex between the hFU homologue and its substrate.

One method exploiting this approach would include the attachment of the hFU substrate to a support and the subsequent incubation of the substrate with the hFU protein, or a biologically active fragment thereof, such as a phosphorylation domain, in the presence of the test compound. The formation of the complex between the substrate and the hFU homologue is then measured.

In a typical test of this kind, a 96-well microtiter plate is coated with the hFU substrate, filled with a plastic scintillator film and incubated with the hFU enzyme, ³²P γ-ATP and different concentrations of the compound to be tested. The radioactivity-labelled phosphate moiety is transferred by the kinase hFU to the substrate coating the wells; after washing the plate, light emission can be directly measured in a scintillation counter. Absence of light emission is indicative of an effective binding of the test compound to the hFU homologue.

Another protocol is based on the use of a biotinylated substrate captured on streptavidin-coated plates and incubated with the enzyme hFU, ATP and different concentrations of the tested compounds. Plates are incubated, washed and the inhibiting activity of the compound is detected measuring the phosphorylated peptide concentration with antibodies anti-phosphotyrosine HRP (Horse Radish Peroxidase) conjugated.

We can also indirectly measure the inhibition of the hFU kinase activity by monitoring spectrophotometrically the decrease in absorbance at 310 nm, due to the NADH oxidation by lactate dehydrogenase and pyruvate, coupled by the hFU enzimatically produced ADP.

Purified hFU can also be coated directly onto plates for use in the aforementioned drug screening techniques. In addition, non-neutralizing antibodies can be used to capture the protein and immobilize it on the solid support.

This invention also contemplates the use of competitive screening assays in which neutralizing antibodies capable of binding hFU specifically complete with a test compound for binding to hFU homologues. In this manner, the antibodies can be used to detect the presence of any peptide which shares one or more antigenic determinants. Thus, the invention provides a method for screening for a compound which shares one or more antigenic determinants with an hFU homologue which comprises providing a screening assay in which neutralizing antibodies capable of binding the hFU homologue specifically compete with a test compound for binding to the hFU homologue.

The inventive purified hFU homologue is a research tool for identification, characterization and purification of interacting, signal transduction pathway proteins. Appropriate labels are incorporated into hFU by various methods known in the art and hFU is used to capture interacting molecules. For example, labelled hFU molecules are incubated with proteins, membranes and all the organelles obtained after cellular lysis, further fractionated by centrifugation on density gradients, to remove unbound molecules, and the hFU complex is quantified. Data obtained using different concentrations of hFU are used to calculate values for the number, affinity, and association of hFU with the signal transduction complex.

Affinity purification may prove to be particularly suitable to this purpose. With this technique, hFU is covalently coupled to a chromatography column. Cells and their membranes are extracted, hFU is removed and various hFU-free subcomponents are passed over the column. Molecules bind to the column by virtue of their hFU affinity. The hFU-complex is recovered from the column, dissociated and the recovered molecule is subjected to N-terminal protein sequencing. This amino acid sequence is then used to identify the captured molecule or to design degenerate oligomers for cloning its gene from an appropriate cDNA library.

Alternatively, compounds may be identified which exhibit similar properties as the hFU homologues of the invention, but which are smaller and exhibits a longer half time in a human or animal body than hFU. When an organic compound is designed, a molecule according to the invention is used as a “lead” compound. The design of mimetics to known pharmaceutically active compounds is a well known approach in the development of pharmaceuticals based on such “lead” compounds. Mimetic design, synthesis and testing are generally used to avoid randomly screening a large number of molecules for a target property. Furthermore, structural data deriving from the analysis of the deduced amino acid sequences encoded by the DNAs of the present invention are useful to design new drugs, more specific and therefore with a higher pharmacological potency.

Comparison of the DNA sequences of the present invention with the sequences present in all the available data bases showed a significant homology with the Drosophila fused cDNA. Computer modelling could therefore be used to develop a putative tertiary structure of the proteins of the invention based on the available information of the Drosophila Fused protein. This approach can help in designing novel enzyme inhibitors based on the predicted structure of the hFU homologues.

In a particular embodiment, the novel molecules identified by the screening methods according to the invention are low molecular weight organic molecules, in which case a pharmaceutical preparation may be prepared thereof for oral intake, such as in tablets.

The pharmaceutical preparations according to the invention may however be prepared for any route of administration, e.g oral, intravenous, cutaneous or subcutaneous, nasal, intramuscular or intraperitoneal. The nature of the carrier or other ingredients will depend on the specific route of administration. (Examples of techniques and protocols that are useful in this context are inter alia found in Remington's Pharmaceutical Sciences, 16^{th} edition, Osol, A (ed.), 1980.)

The dosage of these low molecular weight compounds will depend on the disease state or condition to be treated and other clinical factors such as weight and condition of the human or animal and the route of administration of the compound. For treating human or animals, between approximately 0.5 mg/Kg of body weight to 500 mg/Kg of body weight of the compound can be administered.

The present compounds and methods are advantageously used in order to treat and cure, or prevent all kind of Nevoid Basal Cell Carcinoma Syndrome related disorders and/or diseases, such as basal cell carcinomas. In a particular embodiment, the present molecules are used in gene therapy. For a review of gene therapy procedures, see e.g. Anderson, Science (1992) 256:803-813.

A further advantageous use of the present invention is to develop methods of regulating the signalling of the patched receptor in the body and therefore, the invention also relates to methods of treating a human or animal patient suffering from basal cell carcinomas as well as to methods preventing such conditions.

The cloned genes of the present invention, and oligonucleotides derived therefrom, are useful for screening for restriction fragment length polymorphism (RFLP) associated with certain disorders.

Oligonucleotides derived from the DNA sequences of the present invention encoding the hFU homologues are useful as diagnostic tools for probing the fused gene expression in various tissues. For example, tissue can be probed in situ with oligonucleotide probes carrying detectable groups by conventional autoradiography techniques to investigate native expression of this enzyme or pathological conditions relating thereto.

The present invention is explained in greater detail in the following examples. These examples are intended to be illustrative of the present invention, and should not be construed as limiting thereof.

In the accompanying drawing:

FIG. 1 represents a schematic illustration of the nucleic acid molecules of the invention, which are better described in the following Example 1. With ATG and TGA we have indicated the start and stop codons respectively. Open or solid delta symbols indicates a deletion of nucleic acid sequences. The open box at the left end of the SEQ ID NO.3 scheme indicates the 5′ untranslated region of clone N 2515662; dashed boxes indicate 3′ untranslated regions; solid boxes indicate the kinase domains and squared boxes indicate the remaining part of the coding sequences.

### EXAMPLE 1

This example reports the isolation of the hfu DNA sequences.

Mining the Incyte LifeSeq^{R} database with the use of a cDNA sequence encoding the Drosophila Fused protein (Gen bank Acc X80468) two different clones, one (N 2515662) from a liver tissue cDNA library (LIVRTUTO4), and a second one (N 3168354) from a breast tissue cDNA library (BRSTNOT18), were identified.

Only a partial sequence of the two clones was provided by the Incyte Life Seq^{R} database and these two short sequences were aligned to the Drosophila fused sequence (Gen bank Acc X80468) using the BLAST algorithm. The partial sequence, 274 base pairs (bp) long (SEQ. ID NO.1), associated with the clone N 2515662, was aligned with the Drosophila fused coding sequence (Gen bank Acc X80468) giving an identity score of 65%.

The second partial sequence, 251 bp long (SEQ. ID NO-2), from the clone N 3168354, was aligned with the Drosophila fused coding sequence giving an identity score of 44%. The cDNA from the clone N 2515662 was isolated and both strands were automatic sequenced, providing a full length sequence 4845 bp long (SEQ. ID NO.3). Analogously, both the coding and the complementary strands of the cDNA isolated from the clone N 3168354, were completely sequenced providing a full length sequence 4237 bp long (SEQ. ID NO.4). Both SEQ TD NO. 3 and SEQ ID NO. 4 are depicted in FIG. 1.

From the sequence analysis of the SEQ. ID NO-3 an ATG starting codon was located at position 126, whereas the sequence between position 126 and 835 encodes a deleted kinase domain. Indeed, the alignment of SEQ ID NO-3 and SEQ ID NO.4 showed a deletion of 94 bp between positions 809 and 810 of clone N 2515662, which is illustrated in FIG. 1 by an open delta symbol. This deleted region was replaced by us with the corresponding region of SEQ ID NO. 4 (from nucleotide position 246 to nucleotide position 340) thus yielding a recombinant consensus sequence of 1425 bp (SEQ. ID NO.5) coding for a deduced protein 474 amino acid long (SEQ ID NO.6). See FIG. 1.

This protein exhibits an amino acid identity score with the Drosophila Fused protein (GenBank Acc X80468) of 34,6% and a similarity score of 43,31%; within the kinase domain (aa 1-268) the identity score increases to 51,86% and the similarity score to 63,43%.

From the sequence analysis of SEQ ID NO.4 and its comparison with the sequence of clone N 2515662, it was found a deletion of the ATG starting point and of the 5′ sequence encoding part of the kinase domain, 438 bp long. See FIG. 1.

This deleted region was replaced by us with the corresponding region between position 126 and 563 of the SEQ. ID NO.3 thus yielding an open reading frame (SEQ. ID NO.7) 3948 bp long. See FIG. 1.

The new putative splicing variant was aligned with the predicted SEQ ID NO.5. The alignment of the two cDNAs (SEQ-ID NO.5 and SEQ.ID NO.7) showed a deletion of 103 bp in SEQ ID NO.7 between positions 1378 and 1379. We have indicated the deleted region with a solid delta symbol in FIG. 1.

The translation of the new recombinant consensus sequence (SEQ.ID NO.7), predicts a protein 1315 amino acid long (SEQ.ID NO.8). The deduced aminoacid sequence (SEQ.ID NO.8) was aligned with the Drosophila Fused protein (GenBank Acc X80468) exhibiting an amino acid identity score of 26,93% and a similarity score of 34,67%; within the kinase domain the identity increases to 51,86% while the similarity increases to 63,43%.

### EXAMPLE 2

### Northern Analysis

To study the expression of the putative human homologues of fused gene different Northern Blots were performed and hybridised with the probe herein described.

The sense orientation oligonucleotide 5′-TCAGGCCTATAAACGCATGG-3′ (SEQ ID NO 9) with the antisense-orientation oligonucleotide 5′-CACCTGCAGGCTGTCACTT-31 (SEQ ID NO 10) were used as primers to amplify a portion of the SEQ.ID NO.5 representing the 3′ cDNA coding sequence and the first part of the untranslated sequence after the TGA codon. A fragment 863 bp long was amplified and used as a probe.

Multiple tissue northern blots from Clontech (Human II # 7767-1 and Human fetal # 7756-1) were hybridised with the probe.

Prehybridisation was carried out at 42° C. for 4 h in 5×SSC, 1× Denhardt's, 0.1% SDS, 50% formamide, 250 mg/ml salmon sperm DNA. Hybridisation was performed overnight at 42° C. in the same mixture with the addition of about 1.5×10⁶ cpm/ml of labelled probe.

The probe was labelled with (³²P) dCTP by multiprime DNA labelling system (Amersham), purified on Nick Column (Pharmacia) and added to the hybridizing solution. The filters were washed several times at 42° C. in 0.2×SSC, 0.1 SDS. Filters were exposed to Kodak X-AR film (Eastman Kodak Company, Rochester, N.Y., USA) with intensifying screen at −80° C.

A 5.8 kb mRNA was expressed in testis while a faintest band was detected in fetal lung tissue. Two bands of about 5.8 kb and about 3 kb at equal abundance were detected in mRNA extracted from fetal brain. A 2.4 kb mRNA was expressed at significant level in fetal liver while two bands of about 5.8 kb and 2.4 kb at a relative abundance of about 4:1 were detected in mRNA extracted from fetal kidney.

Equal loading of all the lanes was verified by filter hibridization with a human GAPDH probe.

### EXAMPLE 3

### Polymerase Chain Reaction

The alignment of the two human sequences revealed the presence of two different splicing variants characterised by the insertion of 103 bp outside the functional domain; moreover the two sequences of the invention are characterised by a complete kinase domain without the deletion 94 bp long (see above for details). In order to confirm these two splicing variants 1 ng of template cDNA (Human testis and fetal liver Clontech QUICK-Clone™ cDNAs) was combined with two different PCR-amplification mixes. In the first sample (mixA) the sense orientation oligonucleotide 5′-GTGGAGGAGCGACCATACGA-3′ (SEQ ID NO. 11) and the antisense-orientation oligonucleotide 5′-CCTTTAGGACCTGAAGTTCTGC-G-3′ (SEQ ID NO. 12) were used as primers in the PCR reaction in order to detect the existence of hfu cDNA sequences without the deletion inside the kinase domain. The amplification was carried out in the condition set forth above and the two specific amplification products 343 bp and 249 pb long confirmed the presence of the cDNA sequences with and without the deletion of 94 bp into the functional domain.

In the second sample (mixB) the sense orientation oligonucleotide 5′-CAGCGCATCCAGAGTCAGC-3′ (SEQ ID NO. 13) and the antisense-orientation oligo-nucleotide 5′-CCGGCAGAAGGAATACAAGG-3′ (SEQ TD NO. 14) were used as primers in the PCR reaction in order to amplify the insertion of 103 bp outside the functional domain (see above for details). The amplification was carried out in the condition set forth above and the two specific products of amplification, 253 bp and 150 bp long, confirmed the presence of the insertion of 103 bp into the coding clone.

For each sample the template cDNA was combined with 35 picoMolar of each specific primer, 5 ml of 10× Taq polymerase buffer (500 mM KCl/100 mM Tris-HCl, pH 8.3) 4 ml of 25 mM MgCl₂, 4 ml of a dNTP solution (2.5 mM dNTP) and 0.5 ml (2.5 units) of Taq Gold DNA polymerase (Perkin Elmer Cetus). The reaction volume was brought to 50 ml with H₂O.

The tubes were heated for 10 Min at 95° C., denaturation was carried out for 15 Sec at 95° C., annealing for 30 Sec at 55° C. and polymerization for 30 Sec at 72° C.

The cycle was repeated 40 times.

The two different PCR amplification mixtures amplified both splicing variants. More specifically: using the mix A the amplification products were two bands 343 bp and 249 bp long representing the hfu cDNAs with and without the deletion 94 bp long within the kinase domain. On the other hand, the amplification products obtained using the mixB (two bands 253 bp and 150 bp long), confirmed the existence of the two hfu cDNAs splicing variants with and without the TGA triplet encoding the proteins represented by the SEQ.ID Nos 6 and 8.

1. An isolated single or double stranded polynucleotide which comprises a sequence selected from the group consisting of:
(a) the sense sequence of SEQ.ID NO.5 and the sense sequence of SEQ.ID NO.7;
(b) sequences complementary to the sequences of (a);
(c) a sequence that encodes a polypeptide encoded by the sequences of (a);
(d) analogous sequences that hybridize under stringent conditions to the sequences of (a) and (b); and
(e) sequences that are homologous to the sequences of (a) and that encode polypeptides having hFU activity.

2. The polynucleotide of claim 1 that is a DNA molecule.

3. The polynucleotide of claim 1 that is a RNA molecule.

4. The polynucleotide of claim 2 wherein the nucleotide sequence is SEQ ID NO. 5 or SEQ ID NO. 7.

5. A vector comprising a polynucleotide as defined in any one of claims 1 to 4.

6. A vector according to claim 5, which vector is a plasmid.

7. A vector according to claim 5, which vector is a virus.

8. A host cell transformed with a vector according to claim 5.

9. A transformed host cell according to claim 8, which cell is a bacterial cell.

10. A transformed host cell according to claim 8, which cell is a yeast cell.

11. A transformed host cell according to claim 8, which cell is an insect cell.

12. A transformed host cell according to claim 8, which cell is a mammalian cell.

13. An isolated and purified protein which is coded for by a polynucleotide according to any one of claims 1 to 4.

14. The protein of claim 13 comprising an amino acid sequence having at least approximately 80% sequence homology, preferably approximately 90% sequence homology, more preferably approximately 95% sequence homology and most preferably approximately 98% sequence homology to SEQ ID Nos. 6 or 8.

15. The protein of claim 14 which comprises the amino acid sequence of SEQ ID NO. 6.

16. The protein of claim 14 which comprises the amino acid sequence of SEQ ID NO. 8.

17. A nucleotide probe comprising a sequence of at least 10 contiguous nucleotides of SEQ ID Nos. 5 or 7.

18. A method for screening for a compound which can affect hFU signal transduction comprising:
contacting such a compound with an hFU homologue according to any one of claims 13 to 16; and
assaying for the presence of a complex between the compound and the hFU homologue.

19. A method for screening for an agent that selectively inhibits the hFU kinase activity, comprising assaying a potential agent for the ability to inhibit said hFU kinase activity and not inhibit an activity of another kinase.

20. A method for screening for a compound which shares one or more antigenic determinants with the hFU homologues according to any one of claims 12 to 26 comprising providing a screening assay in which neutralizing antibodies capable of binding the hFU homologue specifically compete with a test compound for binding to the hFU homologue.

21. A method for assaying the ability of a test compound to inhibit the formation of the complex between an hFU homologue and its substrate, comprising
attaching the hFU substrate to a support;
incubating the hFU substrate with the hFU homologue or a biologically active fragment thereof in the presence of the test compound; and
measuring the formation of the complex between the substrate and the hFU homologue.

22. A method according to claims 18 to 21 in which the hFU homologue is labelled.

23. A compound identified through the screening methods of any one of claims 18 to 22.

24. An antibody or antibody fragment which specifically binds a protein according to any one of claims 13 to 16.

25. An antibody according to claim 24 which is a monoclonal antibody or fragment thereof.

26. The antibody of claims 24 or 25 for use in a method of treatment of the human or animal body by therapy.

27. A recombinant cell expressing the antibody according to claims 24 or 25.

28. The protein or compound of any one of claims 13 to 16 and 23 for use in a method of treatment of the human or animal body by therapy.

29. The protein or compound according to claim 28 for use in a method of treatment of a basal cell carcinoma.

30. Use of the protein or compound according to any one of claims 13 to 16 and 23 in the manufacture of a medicament for use in the treatment of basal cell carcinomas.

31. A pharmaceutical preparation comprising a protein or compound according to any one of claims 13 to 16 and 23 together with a pharmaceutically acceptable carrier.

32. A method of treating a host suffering from a basal cell carcinoma, which method comprises administering to the host cell a therapeutically effective amount a protein or compound according to any one of claims 13 to 16 and 23.
